SEQUENCE LISTING (3 sequences)

SEQ ID NO: 1 - protein, 28 aa, mammalian
NGVFKYRPRYFLYKHAYFYPPLKRFPVQ

SEQ ID NO: 2 - protein, 15 aa, mammalian
NGVFKYRPRYFLYKZ

SEQ ID NO: 3 - protein, 15 aa, mammalian
ZHAYFYPPLKRFPVQ

(19) United States
(12) Patent Application Publication
(10) Pub. No.: US 2009/0005298 A1
(43) Pub. Date: Jan. 1, 2009

(54) Bone Sialoprotein Collagen-Binding Peptides

(75) Inventors: Harvey A. Goldberg, London (CA); Graeme K. Hunter, London (CA); Coralee E. Tye, London (CA)

Correspondence Address: THE NATH LAW GROUP, 112 South West Street, Alexandria, VA, 22314, US

(73) Assignee: THE UNIVERSITY OF WESTERN ONTARIO, London, ON (CA)

(21) Appl. No.: 11/660,276

(22) PCT Filed: Feb. 25, 2005
(86) PCT No.: PCT/CA05/00322

§ 371 Date: Jul. 31, 2008

Related U.S. Application Data
(60) Provisional application No. 60/547,788, filed on Feb. 27, 2004.

(51) Int. Cl. A61K 38/00; C07K 14/00; A61P 19/00

(52) U.S. Cl. 514/12; 530/324; 514/14

(57) ABSTRACT

The present invention provides Novel collagen-binding peptides of bone sialoprotein (BSP). Peptides comprising a portion of the N-terminal collagen binding domain of BSP (residues 1-100) are used to stimulate mineralization, nucleate hydroxyapatite, and promote bone formation in collagen expressing tissues. Medicaments for use in the same are also contemplated. Chimeric and conjugate peptides comprising said collagen-binding BSP peptides are also included.

### FIELD OF THE INVENTION

The invention relates to novel collagen-binding peptides. In particular, the invention relates to collagen-binding peptides derived from bone sialoprotein (BSP). The collagen-binding BSP proteins of the invention, or functional analogues thereof, are used to promote or inhibit mineralization of tissue. The invention further relates to pharmaceutical compositions of these peptides, as well as therapeutic uses of such peptides for the treatment of disorders of collagen type I-expressing tissues.

### BACKGROUND OF THE INVENTION

Bone sialoprotein (BSP) is a highly post-translationally modified protein that is expressed in mineralized tissues such as bone, dentin and hypertrophic cartilage. BSP is a phosphorylated sialoprotein of 300 amino acids, containing sulfated tyrosine residues, an RGD (Arg-Gly-Asp) cell-attachment sequence and high contents of acidic amino acids (Asp and Glu) (Ganss et al., 1999, Bone Sialoprotein, Crit Rev Oral Biol Med. 10(1), 79-98). BSP has a MW of approximately 57 kDa, of which approximately 30% is carbohydrate and contains up to 5 moles of phosphate/BSP (Fisher et al., 1983, Matrix sialoprotein of developing bone. J Biol Chem 258, 12723-12727; Franzen and Heinegard, 1985, Isolation and Characterization of two sialoproteins present only in bone calcified matrix, Biochem J 232, 715-724; Wuttke et al., 2001, Structural characterization of human recombinant and bone-derived bone sialoprotein. Functional implications for cell attachment and hydroxyapatite binding. Journal of Biological Chemistry 276, 36839-36848; Zaia et al., 2001, Posttranslational modifications to human bone sialoprotein determined by mass spectrometry. Biochemistry 40, 12983-12991). In mineralized tissues, BSP expression is localized to areas of de novo bone formation and new mineral foci (Bianco et al., 1991, Expression of bone sialoprotein (BSP) in developing human tissues. Calcif Tissue Int 49, 421-426; Chen et al., 1991, Immunohistochemical localization of bone sialoprotein in foetal porcine bone tissues; comparisons with secreted phosphoprotein 1 (SPP-1, osteopontin) and SPARC (osteonectin). Histochem J 23, 281-289). By in situ hybridization, BSP mRNA is found in osteoblasts actively engaged in bone formation, and is not found (or is found only at low levels) in other regions of mineralized tissues (Bianco et al., 1991, Expression of bone sialoprotein (BSP) in developing human tissues. Calcif Tissue Int 49, 421-426; Bianco et al., 1993, Localization of bone sialoprotein (BSP) to Golgi and post-Golgi secretory structures in osteoblasts and to discrete sites in early bone matrix. J Histochem Cytochem 41, 193-203; Chen et al., 1994 Bone sialoprotein mRNA expression and ultrastructural localization in fetal porcine calvarial bone; comparisons with osteopontin. Histochem J 26, 67-78; Chen et al., 1993, Developmental expression of osteopontin (OPN) mRNA in rat tissues; evidence for a role for OPN in bone formation and resorption. Matrix 13, 113-123; Hultenby et al., 1994, Distribution and synthesis of bone sialoprotein in metaphyseal bone of young rats show a distinctly different pattern from that of osteopontin. Eur J Cell Biol 63, 230-239; Riminucci et al., 1995, The anatomy of bone sialoprotein immunoreactive sites in bone as revealed by combined ultrastructural histochemistry and immunohistochemistry. Calcif Tissue Int 57, 277-284). BSP has been shown to be involved in cell attachment, cell signalling, hydroxyapatite (HA) binding, HA nucleation and collagen binding. BSP is thought to serve a role in the mineralization process by acting as a nucleator of hydroxyapatite crystals. Understanding the interaction between BSP and collagen is important as the matrix-mineral relationship in bone is characterized by the presence of HA crystals in the hole zones of the collagen fibrils and by their preferential orientation parallel to the fibril axes (Fratzl P. et al. (1991) Calcified Tissue International 48. 407-413; Fratzl P. et al., (1996) Connect. Tissue Res. 34, 247-287; Weiner S. et al., (1991) FEBS Letters 285, 49-54). To account for such a pattern of mineral deposition it is believed that BSP, shown to be a potent nucleator of HA in vitro (Tye C. E. et al., (2003) J. Biol. Chem. 278; Hunter G. K. et al., (1993) Proc. Natl. Acad. Sci. USA 90, 8562-8565; Harris N. L. et al., (2000) Bone. 27, 795-802), is closely associated with type I collagen fibrils. Binding of BSP to collagen may also be important in mediating cell adhesion to the mineralized matrix. The RGD integrin-recognition sequence of BSP has been postulated to facilitate adhesion of numerous cell types (Stubbs J. T. et al., (1997) J Bone & Min Res 12, 1210-1222; Mintz K. P. et al., (1993) J Bone & Min Res 8, 985-995; Horton M. A. et al., (1995) Ann NY Acad Sci 760, 190-200) to either HA or collagen in the mineralized matrix.

BSP is associated with the demineralized and guanidine-extracted collagenous matrix of bone (Gerstenfeld et al., 1994, Selective extractability of noncollagenous proteins from chicken bone. Calcif Tissue Int 55, 230-235; Kasugai et al., 1992, Temporal studies on the tissue compartmentalization of bone sialoprotein (BSP), osteopontin (OPN), and SPARC protein during bone formation in vitro. J Cell Physiol 152, 467-477), implying a relationship between BSP and collagen. It has been shown that BSP interacts with reconstituted fibrillar collagen (Chen et al., 1992, Calcium and collagen binding properties of osteopontin, bone sialoprotein, and bone acidic glycoprotein-75 from bone. J Biol Chem 267, 24871-24878; Fujisawa and Kuboki, 1992, Affinity of bone sialoprotein and several other bone and dentin acidic proteins to collagen fibrils. Calcif Tissue Int 51, 438-442) and delays collagen fibrillogenesis in a manner similar to that of decorin (Fujisawa and Kuboki, 1992, Calcif Tissue Int 51, 438-442).

The basis of the BSP-collagen interaction is not known. Due to the highly anionic character of BSP, binding to collagen has been proposed to involve non-specific electrostatic interactions (Fujisawa R. and Kuboki Y (1992) Calcified Tissue International 51, 438-442). Studies have demonstrated that BSP isoforms enriched either in phosphate or sulfate may have differing affinities for collagen and mineral crystals (Zhu X. L. et al., (2001) Biochemistry & Cell Biology 79, 737-746). The Applicant has demonstrated that the numerous post-translational modifications of BSP are not involved in collagen binding as native rat bone and prokaryotically-expressed BSP bind collagen with similar affinities (Tye et al; 2005 Identification of the type I collagen-binding domain of bone sialoprotein and the mechanism of interaction. J Biol Chem. 2005 Feb. 8; [Epub ahead of print]). This, along with the lack of binding seen with other acidic macromolecules, such as osteopontin and synthetic homopolymers of poly-glu, suggests a more yet undefined interaction between BSP and collagen (Tye C. E. et al., (2004) Proceedings of the International Conference on the Chemistry and Biology of Mineralized Tissues (ICCBMT), to be published by University of Toronto Press).

U.S. Pat. No. 5,876,454 is directed to the modification of implant surfaces using bioactive conjugates such as BSP. U.S. Pat. No. 6,458,763 is directed to the use of a purified BSP for repair of damaged or diseased bone. U.S. Pat. No. 6,165,487 is directed to tissue engineering for cartilage and bone using a non-immunogenic matrix and a growth factor such as BSP. While these patents disclose the various common uses of BSP, none of these patents has identified any active domain responsible for collagen binding. As such, none of these patents has disclosed the use of any binding characteristics of the BSP protein that could lead to improved therapies for connective tissue diseases/abnormalities.

There is therefore a need to elucidate the interaction between BSP with collagen in a manner to provide novel methods for use in vitro, in vivo, or ex vivo for effectively promoting or inhibiting formation of tissues expressing type I collagen.

### SUMMARY OF THE INVENTION

The Applicant has now identified a novel collagen-binding sequence in bone sialoprotein (BSP) that elucidates the mode and site of interaction of BSP and collagen, and more specifically, collagen type I. This collagen-binding sequence in BSP is localized to an N-terminal portion of the 300-amino acid sequence of the BSP protein and in particular, is localized to amino acid residues 19-46. This collagen-binding BSP sequence is essentially conserved in all species including human and has no apparent homology with previously described peptides that have any collagen-binding properties. With the knowledge of this novel BSP collagen-binding sequence, the peptide can be isolated or synthesized and used directly in a variety of therapeutic methods to treat abnormalities of bone and other connective tissues.

The present invention provides novel collagen-binding BSP peptides, fragments thereof, and functional analogues thereof which can be used in a variety of manners that include but are not limited to the stimulation or inhibition of mineral formation in bone tissue in vitro, in vivo or ex vivo and for tissue engineering applications. Functional analogues of the novel peptides contain one or more amino acid additions, substitutions or deletions to the disclosed peptide sequences. Functional analogues of the collagen-binding BSP peptides of the invention also encompass collagen-binding BSP peptides conjugated to other bioactive agents as well as chimeric collagen-binding BSP peptides. In these aspects the collagen-binding BSP peptide of the invention, provided in conjunction with another bioactive agent or as a chimeric protein, is used as a “carrier” for the delivery of a bioactive agent or other protein/peptide sequence. Depending on the nature of the bioactive agent or other protein/peptide sequence, such collagen-binding BSP peptide “carriers” can be used in compositions and methods to inhibit or stimulate mineralization of a tissue expressing type I collagen.

According to an aspect of the present invention is a collagen-binding BSP peptide comprising a portion of the N-terminal domain of the BSP protein. The BSP protein sequence may be selected from any one of the rat, mouse, human, cow, pig and hamster BSP protein.

According to another aspect of the present invention is a collagen-binding BSP peptide having up to approximately 28 amino acids, the peptide binding to collagen.

According to still another aspect of the invention is a fragment of the collagen-binding BSP peptide, said fragment binding to collagen. Fragments may comprise about 4 amino acids up to the full length of the collagen-binding BSP peptide.

According to another aspect of the present invention is a collagen-binding BSP peptide having the amino acid sequence NGVFKYRPRYFLYKHAYFYPPLKRFPVQ, as well as fragments and functional analogues thereof.

According to another aspect of the present invention is a collagen-binding BSP peptide having an amino acid sequence selected from the group consisting of NGVFKYRPRYFLYK-Z, Z-HAYFYPPLKRFPVQ and NGVFKYRPRYFLYKHAYFYPPLKRFPVQ, as well as fragments and functional analogues thereof.

According to another aspect of the present invention is a chimeric protein that stimulates mineralization of tissues, the chimeric protein comprising:
a collagen-binding BSP peptide; and
a HA-nucleating moiety.

In a further aspect of the invention, there are provided compositions comprising the collagen-binding BSP peptides of the invention, said compositions comprising an effective amount of NGVFKYRPRYFLYKHAYFYPPLKRFPVQ, fragments thereof and/or functional analogues thereof together with a pharmaceutically acceptable diluent or carrier. Such compositions may be formulated to contain additional adjuvant(s), co-stimulatory molecules and/or stabilizers.

In yet a further aspect of the invention, there are provided compositions for stimulating mineralization of a tissue in a mammal, for example, a human, said compositions comprising;
an effective amount of a peptide selected from the group consisting of NGVFKYRPRYFLYK-Z, Z-HAYFYPPLKRFPVQ and NGVFKYRPRYFLYKHAYFYPPLKRFPVQ, wherein said peptide is conjugated to a bioactive molecule that stimulates mineralization of said tissue. The peptide may also be provided as a chimeric protein containing a protein/peptide sequence that stimulates mineralization of said tissue.

According to another aspect of the present invention is a method for stimulating mineralization of a desired tissue expressing type I collagen, the method comprising administering to said tissue an effective amount of a collagen-binding BSP peptide conjugated to a mineralization-promoting bioactive molecule.

According to still another aspect of the present invention is a method for stimulating mineralization of a desired tissue expressing type I collagen, the method comprising administering to said tissue an effective amount of a chimeric collagen-binding BSP peptide, said chimeric peptide further comprising a protein/peptide that stimulates mineralization in said tissue.

According to yet a further aspect of the invention is the use of a collagen-binding BSP peptide in a medicament for the treatment of disorders involving collagen type I expression. The peptides may be selected from the group consisting of NGVFKYRPRYFLYK-Z, Z-HAYFYPPLKRFPVQ, NGVFKYRPRYFLYKHAYFYPPLKRFPVQ as well as any fragment or analogue of NGVFKYRPRYFLYK-Z, Z-HAYFYPPLKRFPVQ or NGVFKYRPRYFLYKHAYFYPPLKRFPVQ and mixtures thereof.

According to a further aspect of the present invention is a method for stimulating bone formation, said method comprising administering an effective amount of:

(a) a collagen-binding BSP peptide conjugated to a mineralization-promoting bioactive molecule;

(b) a chimeric collagen-binding BSP peptide, said chimeric peptide further comprising a protein/peptide that stimulates mineralization; and

(c) a combination of (a) and (b).

According to still a further aspect of the invention is a method for inhibiting mineralization in a tissue, said method comprising administering an effective amount of;

(a) a collagen-binding BSP peptide;

(b) a collagen-binding BSP peptide conjugated with a bioactive molecule that is an inhibitor of mineralization;

(c) a chimeric protein comprising a collagen-binding BSP peptide and an inhibitory HA-nucleating moiety; and

(d) any combination of (a) to (c).

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### DESCRIPTION OF THE FIGURES

The present invention will be further understood from the following description with reference to the Figures, in which:

FIG. 1 shows the amino acid sequence of rat bone sialoprotein used in the current studies. Amino acid residues 1 and 2 are not found in native BSP and were inserted for cloning purposes. Residues after 303 are those that contain the His-tag and Factor Xa cleavage site. The N-terminal signal peptide is not shown. The sequence as shown contains the following: Based on the consensus sequence of BSP from 6 species (rat, mouse, human, cow, pig and hamster; Ganss, Kim and Sodek. 1999. Bone Sialoprotein. Crit Rev Oral Biol Med 10, 79-98.). Identical sequences are shown in bold lettering; sequences conserved (in 3 or 4 of 6 species) are shown in regular lettering; sequences conserved in only (1 or 2 of 6) are shown in italics. The solid underline shows the peptide rBSP43-101 encompassing the first poly[E] sequence and is referred to as Domain 1. The dashed-underline refers to the second poly[E]-containing sequence, rBSP134-206, and is referred to as Domain 2. Demonstrated phosphorylated sites in human BSP are shown by P. The hydrophobic domain postulated to be site for collagen binding is indicated by H--------H (residues 18-45). Casein kinase 2 treatment of prBSP (prokaryotic expressed recombinant BSP) was shown to phosphorylate T259 and T263 and is indicated as CK2. The N-linked glycosylation sites are shown by N (Zaia et al., 2001. Posttranslational modifications to human bone sialoprotein determined by mass spectrometry. Biochemistry 40, 12983-12991). Numerous O-linked glycosylation sites are in the 210-235 domain.

FIG. 2 shows the alignment of rat and human bone sialoprotein sequences encompassing the collagen-binding BSP peptide domain. The numbering refers to the expressed and secreted protein (without the signal peptide). The start of the comparison of the 28-amino acid sequence of the critical domain of rat and human shows 26 identical residues. The identity of this domain strongly suggests the functional importance of this sequence. The 2 differing residues, residue #29 (conserved substitution) and residue #39 show differences amongst all species. This would indicate that these changes are not likely to be critical in the putative function of this domain in BSP.

FIGS. 3A-C show the effects of pH and ionic strength on rBSP and rBSP(1-100) binding. 3A) Binding of rBSP [▴; -] and rBSP(1-100) [▪; ---] to type I collagen as a function of pH was studied using 50 nM rBSP or rBSP(1-100) with 0.1 M phosphate at pH 7.0, 5.5, 4.0, 3.6 and 2.5 as the buffer. Binding is presented as mean percentage bound±S.E, where 100% is the A₄₉₂ of the protein in buffer at pH 7.0. 3B) Binding of 50 mM rBSP [▴; -] and 50 nM rBSP(1-100) [▪; ---] as a function of ionic strength was studied in 25 mM Tris, pH 7.0 containing 0-1000 mM NaCl. Binding is presented as mean percentage bound±S.E, where 100% is the A₄₉₂ of the protein in buffer with 0 mM NaCl. 3C) Binding of 50 nM rBSP in PBS as a function of increasing CaCl₂ [▴; -], MnCl₂ [▪; ---], and LaCl₃ [, - ••••]. Binding is presented as mean percentage bound±S.E, where 100% is the A₄₉₂ of the protein in PBS.

FIGS. 4A-B show the role of poly-glutamic acid domains in binding. 4A) Collagen-coated wells were incubated with increasing concentrations of 6×His-tagged protein, curves were fitted to a 1:1 binding model and K_{D} values were determined. Binding of rBSP [▴; -], rBSP-pE1,2D [▪; ---] and rBSP-pE1,2A [, ••••] to type I collagen was investigated. Data are presented as mean±S.E. 4B). Competition of biotinylated rBSP by unlabelled proteins: 10 nM biotinylated rBSP was incubated simultaneously with 0-1000 mM of unlabelled rBSP-pE1,2D [▪; ---] and rBSP-pE1,2A [, ••••]. Binding is indicated as mean percentage bound±S.E., where 100% is the absorbance of biotinylated rBSP containing no competitor.

FIGS. 5A-D show the binding of rBSP peptides to type I collagen. 5A) Binding of rBSP(1-100) [▴; -], rBSP(99-201) [▪; ---] and rBSP(200-301) [; ••••] to collagen. Collagen-coated wells were incubated with increasing concentrations of protein and curves were fitted to a 1:1 binding model and K_{D} values were determined. (Note that the curves for rBSP(99-201) and rBSP(200-301) are superimposed). 5B) Competition of 25 nM biotinylated rBSP by unlabelled rBSP [▴; -], rBSP(1-100) [▪; ---], rBSP(99-200) [; ••••] and rBSP(200-301) [♦; -.-.-.]. Binding is indicated as mean percentage bound±S.E., where 100% is the absorbance of biotinylated rBSP containing no competitor. 5C) Binding of rBSP(1-100) [▴; -], rBSP(1-75) [▪; ---] and rBSP(19-100) [; •••] to collagen. 5D) Competition of 50 nM biotinylated rBSP(1-100) by rBSP(1-100) [▴; -], rBSP(1-75) [▪; ---], rBSP(19-100) [♦; •••] and rBSP(43-101) [♦; -.-.-.]. Binding is indicated as mean percentage bound±S.E., where 100% is the absorbance of biotinylated rBSP(1-100) containing no competitor.

FIG. 6 shows the circular dichroism spectra of rBSP(1-75) with decreasing pH. rBSP(1-75) was studied at 0.2 mg/ml in 0.1 M phosphate at pH 7.0 (-); pH 4.0 (---); and pH 2.5 (***).

FIG. 7 is a micro-CT scan at day 33 showing mineral deposition in an in vivo model for bone repair. Six mm ‘critical-sized’ defects were created in calvaiae of adult Wistar rats. An absorbable collagen sponge was immersed in buffer alone (a,b); native rat BSP at 4 μg/sponge (c,d); or prokaryotically expressed recombinant BSP −1-100 peptide at 40 μg/sponge (e,f). rBSP 1-100 contains the collagen-binding domain of the invention and the first Glu-rich domain involved in HA-nucleation.

### DETAILED DESCRIPTION OF THE PREFERRED EMBODIMENTS

The present invention provides novel collagen-binding BSP peptides which are demonstrated to bind type I collagen. These peptides have a variety of uses in compositions and methods to promote mineral formation in tissues containing collagen. In aspects of the invention, the collagen-binding BSP peptides are used to promote mineral formation in hard or soft tissues containing (i.e. expressing) type I collagen (e.g. tendon, bone, dentin, skin, periodontal ligament). To promote mineralization of such tissues, the collagen-binding BSP peptides of the invention can be linked with an appropriate bioactive agent that stimulates mineralization. Alternatively, the collagen-binding BSP peptides may be presented as a chimeric peptide comprising a protein/peptide sequence that promotes mineral formation and thus bone formation both in vitro and in vivo in mammals and in ex-vivo tissue engineering. Such peptides have a variety of uses in connective-tissue repair and regeneration and can be used alone or in further combination with other active agents. It is also within the scope of the present invention to use the collagen-binding BSP peptides of the invention to inhibit mineralization in tissues where such mineralization is inappropriate. In this manner, the bioactive peptide or protein/peptide sequence is selected such that it has the properties of inhibiting mineralization. The peptides of the invention can be used directly or formulated within a variety of compositions, and/or devices to be used to promote or inhibit mineralization as desired.

The Applicant had previously demonstrated type I collagen-binding properties of bone-extracted BSP and prokaryotically-expressed rat recombinant BSP (rBSP) but not osteopontin (OPN) using a solid-phase assay and surface plasmon resonance (BIAcore™) (Tye C. E. et al., (2004) Type I Collagen Interaction with Bone Sialoprotein and Osteopontin, Proceedings of the International Conference on the Chemistry and Biology of Mineralized Tissues (ICCBMT), to be published by University of Toronto Press; the disclosure of which is incorporated herein in its entirety). Based on these analyses, native and rBSP had the highest level of binding to collagen, similar to fibronectin, followed in decreasing order by native OPN and rOPN, and by synthetic poly-glutamic acid, which showed little or no binding. Protein-collagen interactions were also studied by immunogold labelling using anti-BSP and OPN antibodies. A high degree of labelling of both native BSP and rBSP on fibrillar collagen was observed, whereas very little OPN immunoreactivity was evident. The differing binding properties of the acidic protein/peptides used in these studies suggest that BSP exhibits specific binding to collagen. Furthermore, since both native and rBSP exhibit similar binding affinities, it appeared that post-translational modifications were not critical for the binding of BSP to collagen (Tye et al; 2005 Identification of the type I collagen-binding domain of bone sialoprotein and the mechanism of interaction. J Biol Chem. February 8; [Epub ahead of print]).

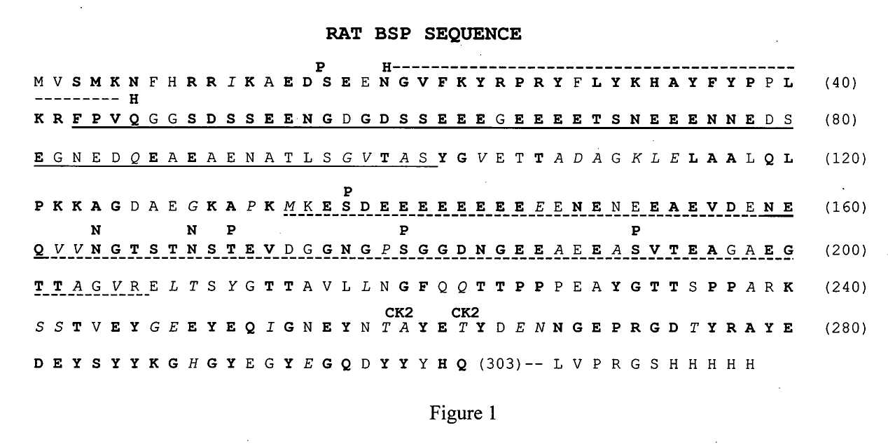

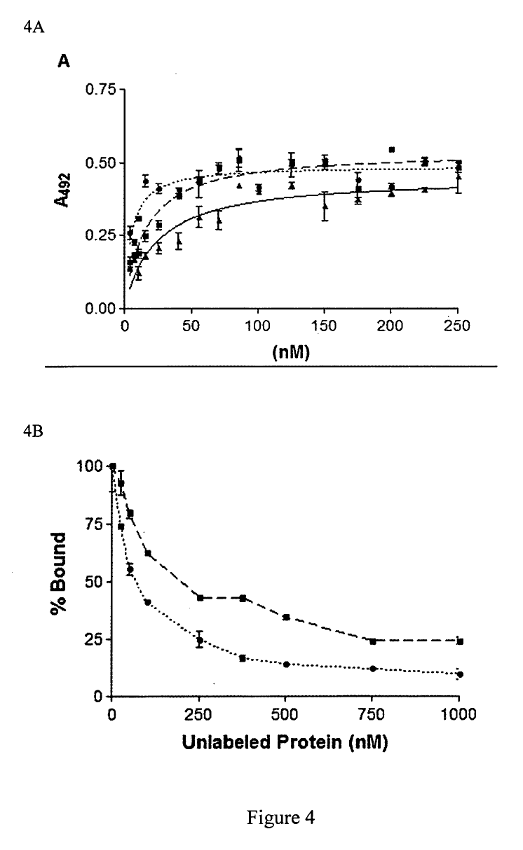

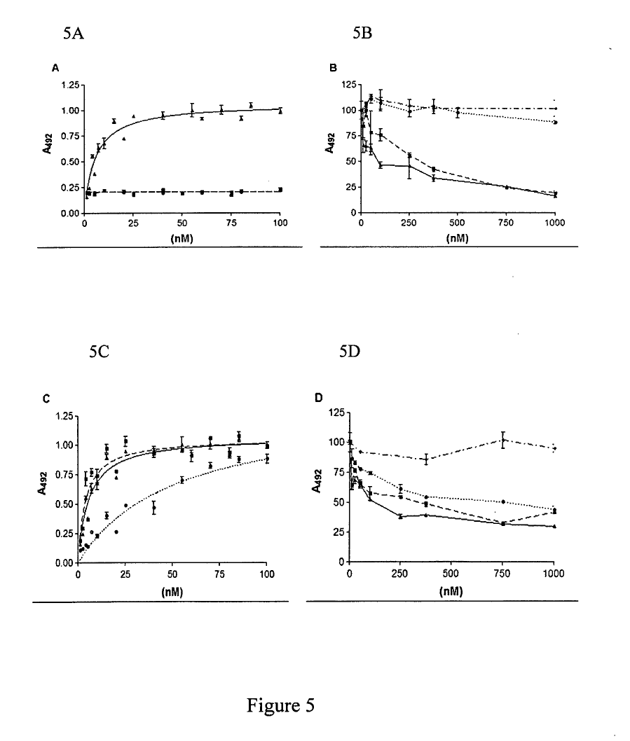

In order to locate the collagen-binding domain within the rBSP sequence, three peptides, each encompassing a third of the protein, were prepared and evaluated for collagen binding activity. The amino acid sequence of rat BSP used in the experiments is shown in FIG. 1. The rBSP (1-100) bound to collagen with high affinity, and competitively inhibited binding of rBSP, indicating the high specificity of the peptide. The rBSP(99-200) and rBSP(200-301), however, demonstrated no affinity for collagen and this was confirmed in the inability to competitively inhibit the binding of rBSP to collagen (FIG. 5A-D). The lack of binding by the poly(E)-containing rBSP(99-200) demonstrated that the poly(E) regions (i.e. those enriched in glutamic acid residues) are not responsible for the binding (FIG. 4A-B).

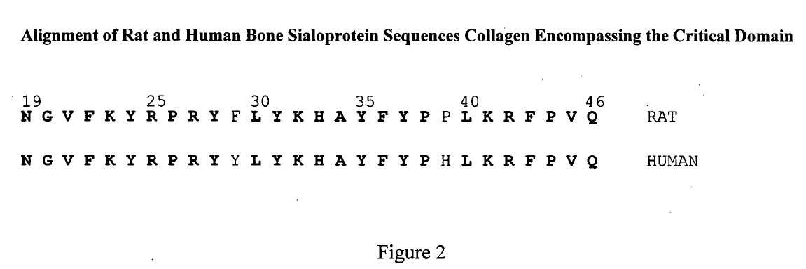

Two smaller N-terminal peptides were tested for collagen-binding activity and it was found that rBSP(1-75) bound to collagen with similar affinity to the rBSP(1-100) peptide, indicating that the binding domain was within the N-terminal 75 residues of BSP. The rBSP(19-100) peptide demonstrated saturable binding to collagen, but with lower affinity than rBSP(1-100). This suggested that the first 19 residues of BSP were somewhat involved in collagen binding but are not entirely necessary to achieve binding. However, the specificity of the rBSP(1-75) and rBSP(19-100) peptides was confirmed by their ability to competitively inhibit the binding of rBSP(1-100) to collagen. A final peptide, rBSP(43-101) was not able to inhibit binding of rBSP(1-100) giving further insight into the location of the collagen binding domain. It therefore appeared that the collagen-binding domain was located within amino acid residues 19-46 (FIG. 2).

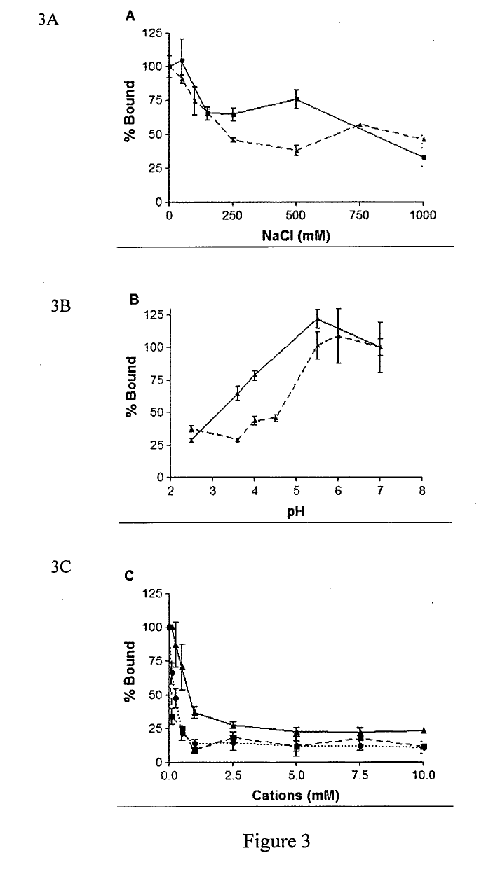

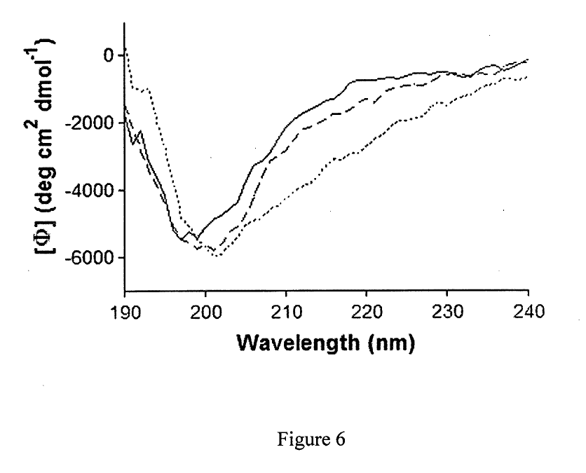

The involvement of electrostatic interactions in the binding of rBSP and rBSP(1-100) to collagen was examined by increasing the ionic strength or decreasing the pH of the buffer used during binding (FIGS. 3A-C). Both of these changes caused a considerable decrease in binding to collagen. While altering pH of the buffer did cause an increase in alpha-helical content of the rBSP(1-75) peptide (FIG. 6), it did not seem that conformational changes were solely responsible for the decrease in binding as no change in conformation was seen with increasing ionic strength. The observed decrease in binding therefore indicated an electrostatic interaction between BSP and collagen. As binding to collagen was never completely abolished, even at very low pH or very high salt, it appeared that electrostatic forces are only a component of the binding mechanism.

The binding of BSP to collagen is calcium-independent as the addition of calcium or manganese or lanthanum, was found to decrease the amount of protein bound to collagen. In vivo, binding of BSP to collagen occurs in a calcium-rich environment. Binding to collagen was never completely abolished by calcium. Again, an electrostatic mechanism of binding was evident as the cations were likely binding weakly to the negatively charged surface of BSP and interfering with the electrostatic interactions involved in collagen binding. However binding was never completely abolished.

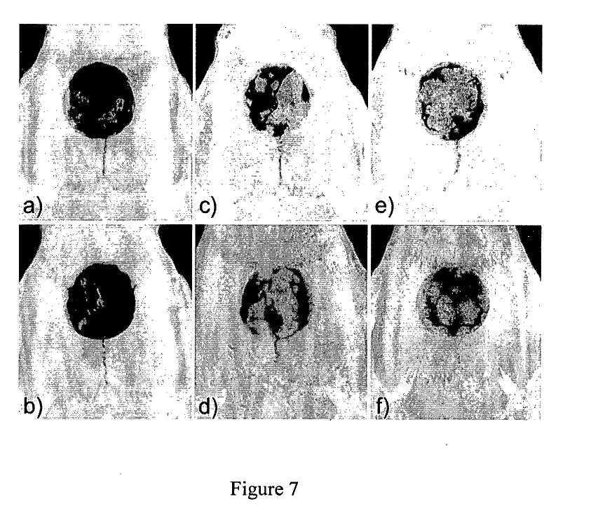

The ability of peptides containing the BSP collagen binding sequence of the invention to repair a bone defect was demonstrated in an in vivo model for bone regeneration. It was demonstrated that the collagen-binding and hydroxyapatite-nucleating activities but not cell-attachment properties of the peptides, initiate repair of a bone defect. The results suggested that mineralization within the collagen gel was occurring at a greater rate with both native rat BSP and the recombinant rBSP-1-100 peptide than that of the no-BSP, vehicle alone controls (FIG. 7). This is of distinct interest in that while it has been shown that BSP can enhance bone repair it has been believed (but not proven) that the main reason for this activity is the cell-attachment domain located in the carboxy-terminal end of the protein (in rat, the Arginine-Glycine-Aspartic acid cell attachment sequence is located at residues 270-272). This demonstration that a recombinant peptide lacking this cell-attachment sequence and post-translational modifications can induce mineral formation in vivo supports the use of the BSP peptides of the invention to repair defects in collagen-rich tissues such as bone.

Additional in vitro experiments were done to compare the efficacy of BSP in promoting mineral formation in the presence of a standard scaffold, agarose gels (Hunter and Goldberg, Proc. Natl. Acad. Sci. USA 90, 8562-8565, 1993) versus reconstituted type I collagen (fibrils). Using a standard definition of potency for hydroxyapatite nucleation (defined in Tye et al; J. Biol. Chem. 278, 7949-7955, 2003) it was demonstrated that recombinant full-length BSP in agarose gels is active at concentrations as low as 0.25 nmol. However, nucleating activity of rBSP in a collagen scaffold was shown with concentrations as low as 0.025 nmol; a 10-fold difference. Thus this also supports the contention that the unique collagen-binding domain in BSP is directly associated with enhancing the potency of a known bioactive property of BSP. This enhancement is likely due to stabilization of the conformation of the protein, which is known to be highly flexible in solution (Tye et al., 2003, above).

The Applicant has now for the first time demonstrated that the residues involved in the non-electrostatic interactions, i.e. collagen-binding, are located within amino acid residues 19-46 (FIGS. 1, 2), with residues on either side of this region being involved in electrostatic interactions. There are five positively charged and four negatively charged amino acid residues within the first 19 amino acid residues of BSP, as well as numerous negatively charged amino acids C-terminal (i.e. downstream) of amino acid residue 46, which may be involved in the long-range electrostatic interactions. Amino acid residues 19-46 (rat: NGVFKYRPRYFLYKHAYFYPPLKRFPVQ) are in a region that is very highly conserved (FIG. 1). Between the rat, mouse, porcine, bovine and human sequences there are 20 identical residues, 4 highly conserved residues, 2 conserved and 1 non-conserved residue. Uncharacteristic of the rest of the sequence of BSP is the lack of negatively charged residues within this region. There are positively charged Lys, Arg and His residues, however, as well as an enrichment of Pro, Tyr and Phe residues.

The collagen-binding BSP peptide of the invention has been identified to have the amino acid sequence NGVFKYRPRYFLYKHAYFYPPLKRFPVQ (Sequence ID No. 1). In aspects of the invention, the peptide may be selected from the group consisting of NGVFKYRPRYFLYK-Z (Sequence ID No. 2) and Z-HAYFYPPLKRFPVQ (Sequence ID No. 3) where these peptides contain one or more additional amino acids at either the N-terminal or carboxy terminal side represented by Z. “Z” may be selected from one or more amino acids such as but not limited to alanine, arginine, asparagines, aspartate, cysteine, glutamate, glutamine, glycine, histidine, isoleucine, leucine, lysine, methionine, phenylalanine, praline, serine, threonine, tryptoph an, tyro sine, valine, hydroxyproline and any other modified amino acid.

It is understood by one of skill in the art that the collagen-binding BSP peptide of the invention may comprise at least four amino acids in length and up to any fragment size of the sequences NGVFKYRPRYFLYKHAYFYPPLKRFPVQ (Sequence ID No. 1), NGVFKYRPRYFLYK-Z (Sequence ID No. 2) and Z-HAYFYPPLKRFPVQ (Sequence ID No. 3). Furthermore, the fragments may be provided at any location within the sequences. The peptide sequences as described herein utilize the standard 1-letter code for amino acids as is understood by one of skill in the art (Short Protocols In Molecular Biology, Second Edition, John Wiley & Sons, 1992).

The present invention also relates to functionally equivalent variants of the peptides as described above and herein. “Functionally equivalent variants” or “functional analogues” include peptides with partial sequence homology, peptides having one or more specific conservative and/or non-conservative amino acid changes, peptide conjugates, chimeric proteins, fusion proteins and peptide-encoding nucleic acids. Functionally equivalent variants also encompasses modified peptides such as phosphorylated peptides where phosphorylated sites are present at one or more amino acids of the peptide sequence. Such modifications may also be provided in the conjugated and chimeric peptides. The functionally equivalent variants maintain the biological activity of the native peptide. The biological activity (i.e. binding to collagen) may be assessed by a collagen-binding assay as described herein and is well within the scope of those of skill in the art.

In terms of “functional analogues”, it is well understood by those skilled in the art, that inherent in the definition of a biologically functional peptide analogue is the concept that there is a limit to the number of changes that may be made within a defined portion of the molecule and still result in a molecule with an acceptable level of equivalent biological activity, which, in this case, would include the ability to induce changes in collagen-binding and in particular, type I collagen-binding. A plurality of distinct peptides/proteins with different substitutions may easily be made and used in accordance with the invention. It is also understood that certain residues are particularly important to the biological or structural properties of a protein or peptide such as residues in the receptor recognition region, such residues of which may not generally be exchanged.

Functional analogues can be generated by conservative or non-conservative amino acid substitutions. Amino acid substitutions are generally based on the relative similarity of the amino acid side-chain substituents, for example, their hydrophobicity, hydrophilicity, charge, size and the like. Thus, within the scope of the invention, conservative amino acid changes means an amino acid at a particular position which is of the same type as originally present; i.e. a hydrophobic amino acid exchanged for a hydrophobic amino acid, a basic amino acid for a basic amino acid, etc. Examples of conservative substitutions include the substitution of non-polar (hydrophobic) residue such as isoleucine, valine, leucine or methionine for one another, the substitution of one polar (hydrophilic) residue for another such as between arginine and lysine, between glutamine and asparagine, the substitution of one basic residue such as lysine, arginine or histidine for another, or the substitution of one acidic residue, such as aspartic acid or glutamic acid for another, the substitution of a branched chain amino acid, such as isoleucine, leucine, or valine for another, the substitution of one aromatic amino acid, such as phenylalanine, tyrosine or tryptophan for another. Such amino acid changes result in functional analogues in that they do not significantly alter the overall charge and/or configuration of the peptide. Examples of such conservative changes are well-known to the skilled artisan and are within the scope of the present invention. Conservative substitution also includes the use of a chemically derivatized residue in place of a non-derivatized residue provided that the resulting peptide is a biologically functional equivalent to the peptide as described herein.

The peptide of the invention may be provided as a chimeric protein such that it contains a protein/peptide amino acid sequence from another protein. In this manner the collagen-binding BSP peptide acts as a “carrier” for the other attached sequence. The protein/peptide amino acid sequence can be selected such that it would stimulate or inhibit mineralization of a tissue as presented/targeted to the tissue by the collagen-binding BSP portion thereof. In one aspect, a chimeric peptide of the present invention may comprise a collagen-binding BSP peptide expressed with another type of binding site such as for example calcium-binding sequences of protein such as osteopontin, osteonectin (SPARC), dentin sialoprotein, dentin matrix protein-1, osteocalcin, phosvitin, phosphosphoryn, beta-casein, stratherin, matrix gla protein, riboflavin binding protein and alpha S1 casein. It is also desirable to provide a chimeric protein comprising a collagen-binding BSP peptide and mineral-nucleating sequence of bone sialoprotein, phosphorphoryn and dentin matrix protein-1. Such mineral-nucleating sequences functioning to stimulate mineralization of a tissue. It is also within the scope of the present invention to provide a chimeric protein comprising the collagen-binding BSP peptide of the present invention and a collagen-binding domain from a known collagen-binding protein such as fibronectin, von Willebrand factor and decorin. A chimeric peptide of the present invention may also comprise the addition of a variety of other peptides/proteins to the collagen-binding BSP sequences of the present invention, such peptides/proteins may include but are not limited to basic fibroblast growth factors, acidic fibroblast growth factors, vascular endothelial growth factors, PD-ECGF, HGF, angiogenin, cell growth factors belonging to the EGF family (TGF-alpha, EGF, SDGF, beta-celluin), PDGF, integrin-alpha/beta, angiopoietin-1, TNF-alpha, IGF, G-CSF, growth hormone, angiogenesis inhibitors, TGF-beta, TGF-alpha, NGF, HGF, CTGF, growth factors in general, BMPs, cytokines, lymphokines, chemokines, interferons, interleukins, colony stimulating factors, erthythropoietin, tumor necrosis factor, insulin, PTH, enzymes (MMPs, streptokinase), platelet factor and combinations thereof. The chimeric proteins of the invention may be produced by recombinant expression of a fusion polynucleotide comprising the collagen-binding BSP peptide sequence and a different desired protein/peptide sequence. Methods for recombinant expression of fusion polynucleotides are well known to those of skill in the art. The nucleotide sequence coding for a chimeric protein, or a functional equivalent, is inserted into an appropriate expression vector, i.e., a vector which contains the necessary elements for the transcription and translation of the inserted coding sequence(s).

Methods which are well known to those skilled in the art can be used to construct expression vectors containing the chimeric protein coding sequence and appropriate transcriptional/translational control signals. These methods include in vitro recombinant DNA techniques, synthetic techniques and in vivo recombination/genetic recombination. See, for example, the techniques described in Sambrook et al., 1989, Molecular Cloning A Laboratory Manual, Cold Spring Harbor Laboratory, N.Y. and Ausubel et al., 1989, Current Protocols in Molecular Biology, Greene Publishing Associates and Wiley Interscience, N.Y.

A variety of host-expression vector systems may be utilized to express the chimeric protein coding sequence. These include but are not limited to microorganisms such as bacteria transformed with recombinant bacteriophage DNA, plasmid DNA or cosmid DNA expression vectors containing the chimeric protein coding sequence; yeast transformed with recombinant yeast expression vectors containing the chimeric protein coding sequence; insect cell systems infected with recombinant virus expression vectors (e.g., baculovirus) containing the chimeric protein coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the chimeric protein coding sequence; or animal cell systems. It should be noted that since most apoptosis-inducing proteins cause programmed cell death in mammalian cells, it is preferred that the chimeric protein of the invention be expressed in prokaryotic or lower eukaryotic cells. The expression elements of each system vary in their strength and specificities. Depending on the host/vector system utilized, any of a number of suitable transcription and translation elements, including constitutive and inducible promoters, may be used in the expression vector.

It is also within the scope of the present invention to have one or more linker molecules attached to the collagen-binding BSP peptides of the invention in order to provide a conjugate of the peptide. In this manner, the collagen-binding BSP peptide of the invention may act as a “carrier” for a bioactive molecule that may be attached via the linker molecule. In aspects, the linker molecules may be attached at either or both sides (i.e. the end portions) of a selected peptide. Such linker molecules may be selected from the group consisting of carbodiimide, aldehyde, maleimide, sulfhydryl, amino, carboxy, hydroxy and NHS esters, a modified cysteine, a phosphorylated amino acid, an amino acid, diacetic acid, sulfonyl chloride, isocyanate, isothiocyanate, epoxy, bisphosphonate, pyrophosphate, phosphate, disulfide, phenyl azide, alkyl halide and hydrazide acyl chloride. Again, the linker molecules can be used to facilitate the binding of desired pharmaceutical agents to the peptides of the invention to allow the delivery of pharmaceutical agents at a desired site. The collagen-binding activity is thus imparted to the desired pharmaceutical agent. Such pharmaceutical agents may include but are not limited to chemotherapeutic agents and agents used in the treatment of diseases involving inappropriate collagen type I expression. Other pharmaceutical agents for use with the collagen-binding BSP peptides of the invention are non-steroidal anti-inflammatory drugs. Alternatively, the pharmaceutical agent may be a therapeutic radionuclide or an imaging agent. Cleavable linker moieties that are specifically chemically or enzymatically cleaved are also encompassed in the invention. Proteolytically cleavable linker molecules, in aspects, may comprise a linker molecule that is a proteinase recognition sequence.

The peptides of the invention may be obtained by chemical synthesis using automated instruments or alternatively by expression from nucleic acid sequences which are capable of directing synthesis of the peptide using recombinant DNA techniques well known to one skilled in the art. The peptides of the invention may be prepared by chemical synthesis using techniques well known in the chemistry of proteins such as solid phase synthesis (Merrifield, J. Am. Chem. Assoc. 85:2149-2154 (1964)) or synthesis in homogenous solution (Houbenweyl, Methods of Organic Chemistry (1987), (Ed. E. Wansch) Vol. 15, pts. I and II, Thieme, Stuttgart). Techniques for production of proteins by recombinant expression are well known to those in the art and are described, for example, in Sambrook et al. (1989) or latest edition thereof. As the sequence for BSP is conserved amongst a variety of species, any nucleic acid sequence from rat, mouse, porcine, hamster, bovine or human sequence may be used in the invention as is understood by one of skill in the art.

The present invention also contemplates non-peptide analogues of the peptides of the invention, e.g. peptide mimetics that provide a stabilized structure or lessened biodegradation. Peptide mimetic analogues can be prepared on the basis of the sequences NGVFKYRPRYFLYKHAYFYPPLKRFPVQ (Sequence ID No. 1), NGVFKYRPRYFLYK-Z (Sequence ID No. 2) and Z-HAYFYPPLKRFPVQ (Sequence ID No. 3), or fragments thereof, by replacement of one or more residues by non-peptide moieties. Preferably, the non-peptide moieties permit the peptide to retain its natural conformation, or stabilize a preferred, e.g. bioactive conformation. Such peptides can be tested in molecular or cell-based binding assays to assess the effect of the substitution(s) on conformation and/or activity. The preparation of non-peptide mimetic analogues from the peptides of the invention can be done, for example, as taught in Nachman et al., Regul. Pept. 57: 359-370 (1995).

The present invention also encompasses nucleic acid molecules comprising a nucleotide sequence which encodes a sequence selected from the group consisting of NGVFKYRPRYFLYKHAYFYPPLKRFPVQ (Sequence ID No. 1), NGVFKYRPRYFLYK-Z (Sequence ID No. 2) and Z-HAYFYPPLKRFPVQ (Sequence ID No. 3) as well as variants thereof.

The present invention also encompasses nucleic acid molecules comprising a nucleotide sequence which encodes a sequence selected from the group consisting of NGVFKYRPRYFLYKHAYFYPPLKRFPVQ (Sequence ID No. 1), NGVFKYRPRYFLYK-Z (Sequence ID No. 2) and Z-HAYFYPPLKRFPVQ (Sequence ID No. 3) as well as fragments and variants thereof. Also encompassed by the present invention are nucleic acid sequences which are complementary as well as anti-complementary to a sequence encoding and equivalent sequence variants thereof. One skilled in the art would readily be able to determine such complementary or anti-complementary nucleic acid sequences. Also as part of the invention are nucleic acid sequences which hybridize to one of the aforementioned nucleic acid molecules under stringent conditions. “Stringent conditions” as used herein refers to parameters with which the art is familiar and such parameters are discussed, for example, in the latest editions of Molecular Cloning: A Laboratory Manual, J. Sambrook, et al., eds., Cold Spring Harbor Laboratory Press, Cold Spring Harbor, N.Y., or Current Protocols in Molecular Biology, F. M. Ausubel, et al., eds., John Wiley & Sons Inc., New York. One skilled in the art would be able to identify homologues of nucleic acids encoding the collagen-binding BSP peptides of the invention. Cells and libraries are screened for expression of such molecules which then are routinely isolated, followed by isolation of the pertinent nucleic acid molecule and sequencing. The invention contemplates the use of the rat, human, mouse, bovine, pig and hamster BSP amino acid and nucleic acid sequences as is understood by one of skill in the art.

It is noted that the nucleic acid molecules described herein may also encompass degenerate nucleic acids. Due to degeneracy in the genetic code, variations in the DNA sequence will result in translation of identical peptides. It is thus understood that numerous choices of nucleotides may be made that will lead to a sequence capable of directing production of the peptides or functional analogues thereof of the present invention. As a result, degenerative nucleotide substitutions are included in the scope of the invention.

As would be understood by one of skill in the art, nucleic acid molecules of the present invention may encompass single and double stranded forms, plasmid(s), viral nucleic acid(s), plasmid(s) bacterial DNA, naked/free DNA and RNA. A viral nucleic acid comprising a nucleic acid sequence encoding for at least one peptide of the invention may be referred to as a viral vector.

The invention also encompasses expression vectors comprising the nucleic acid sequences of the invention encoding one or more of the collagen-binding BSP peptides of the invention and functional analogues thereof within expression vectors. Any expression vector that is capable of carrying and expressing the nucleic acid sequences encoding for the peptides of the invention and functional analogues thereof in prokaryotic or eukaryotic host cells may be used, including recombinant viruses such as poxvirus, adenovirus, alphavirus and lentivirus.

The invention also encompasses host cells transformed, transfected or infected with such vectors to express the peptides or functional analogues of the invention. As such, host cells encompass any potential cell into which a nucleic acid of the present invention may be introduced and/or transfected. The invention further contemplates the production of chimeric proteins comprising the collagen-binding BSP peptides of the invention.

The peptides of the invention (including any fragments and any functional analogues thereof) can be used alone or be provided as a composition to alter mineralization of type I collagen-expressing tissues in vitro, in vivo, or ex vivo. In embodiments of the invention compositions comprising one or more collagen-binding BSP peptides of the invention are linked to bioactive agents or as chimeric peptides that include a HA-nucleating domain sequence in order to possess mineralization-promoting activity that can be used regenerate bone at specific sites for skeletal tissue repair and skeletal tissue engineering. The pharmaceutical compositions of the invention comprising a chimeric protein of a collagen-binding BSP peptide and HA-nucleating domain can be delivered to specific sites to stimulate bone formation to treat skeletal trauma, skeletal development abnormalities (both non-metabolic bone diseases and metabolic bone diseases), arthritis and degenerative joint diseases. Representative uses of the peptides of the present invention when linked with hydroxyapatite-nucleating agents are for bone trauma or bone development abnormalities include for example repair of bone defects and deficiencies, such as those occurring in closed, open and non-union fractures, bone/spinal deformation, osteosarcoma, myeloma, bone dysplasia and scoliosis; prophylactic use in closed and open fracture reduction; promotion of bone healing in plastic surgery; stimulation of bone in-growth into non-cemented prosthetic joints and dental implants; elevation of peak bone mass in pre-menopausal women; treatment of growth deficiencies; treatment of periodontal disease and defects, and other tooth repair processes; increase in bone formation during distraction osteogenesis; and treatment of other skeletal disorders, such as age-related osteoporosis, post-menopausal osteoporosis, glucocorticoid-induced osteoporosis or disuse osteoporosis and arthritis, osteomalacia, fibrous osteitis, renal bone dystrophy and Paget's disease of bone, or any condition that benefits from stimulation of mineralization leading to bone formation. It is understood to one of skill in the art that chimeric collagen-binding BSP peptides comprising a HA-nucleating domain protein sequence can also be used to treat other mineralized tissue defects and connective tissue disorders and in wound repair.

Agents such as bone morphogenetic factors, anti-resorptive agents, osteogenic factors, cartilage morphogenetic factors, growth hormones and differentiating factors may be used together with the collagen-binding BSP peptide compositions of the invention in order to aid in the promotion of bone development, maintenance and repair. The compositions of the present invention can be useful in repair of congenital, trauma-induced or surgical resection of connective tissue (for instance, for cancer treatment), and in cosmetic surgery. Such tissue deficit or defect can be treated in vertebrate subjects by administering the peptides of the invention which exhibit certain structural and functional characteristics.

The peptides of the invention may be used for skeletal reconstruction involving ex vivo tissue engineering of bone tissue for implantation in a vertebrate. Cells and/or developing tissues can be treated in vitro with a selected peptide(s) or functional analogue thereof during the tissue engineering process to promote any or all the steps of cell proliferation, cell differentiation, and/or tissue construct formation. The cell source for the tissue engineering process may be autologous, allogenic, or xenogeneic. The ex vivo process may be concluded after cell expansion, cell differentiation or tissue construct formation, and the cells and/or tissues so produced introduced into the patient.

It is also within the scope of the present invention to use one or more of the peptides of the invention to inhibit mineralization and thus the formation of bone. This is desirable in conditions where HA crystals form in tissues not normally calcified such as in atherosclerotic plaques, soft tissues of patients with abnormally high circulating calcium or phosphate, and articular cartilage of patients with degenerative joint diseases. In this aspect of the invention the collagen-binding BSP peptides of the invention are conjugated to pharmaceutical agents or bioactive molecules that inhibit or decrease tissue mineralization. Alternatively, the collagen-binding BSP peptides are provided as chimeric peptides with a protein/peptide sequence that prevents or inhibits HA-nucleation in order to reduce bone formation and may be further provided in conjunction with a desired pharmaceutical agent(s) and/or bioactive molecule(s) in an individual in need of such treatment. The collagen-binding BSP peptides may also be used to block collagen binding sites to inhibit mineralization in degenerative joint diseases and those diseases involving osteophyte production. Chondrocytes under certain conditions may express type I collagen leading to binding and mineralization. The peptides of the invention may be thus used to prevent overaccumulation of collagen by inhibiting fibrillogenesis and as such be useful in diseases of fibrosis, such as scleroderma and cirrhosis of the liver. The collagen-binding BSP peptides may be conjugated/linked to other bioactive compounds that promote collagen-rich soft connective tissue repair.

The peptides of the invention can be directed conjugated to pharmaceutical agents or provided as chimeric peptides with HA-nucleating domain peptides in order to stimulate mineralization of desired tissues and thus bone formation. It is also within the scope of the invention to further conjugate the chimeric peptides of the invention with a pharmaceutical agent. All forms of the collagen-binding BSP peptide of the invention can be formulation as a composition.

The compositions of the invention may be administered systemically or locally. Local targeted administration is preferred at the site of treatment or tissue injury. For example, the composition may be prepared for local administration by intra-articular injection or use with an implant. For systemic use, the compounds herein are may be formulated for administration selected from intravenously and subcutaneously according to conventional methods. For systemic use, the peptides of the invention may comprise additional targeting molecules as is understood by one of skill in the art.

The compositions of the invention may be in the form of a liquid preparation, or in a solid dispersion, it can be packed in capsules or shaped into pellets, fine granules, granules or tablets. As a solid dispersion, the composition may be shaped into solid forms such as spheres, rods, needles, pellets and films in the presence of additional additives as necessary as is understood by one skilled in the art.

Intravenous administration can be by a series of injections or by continuous infusion over an extended period. Administration by injection or other routes of discretely spaced administration can be performed at intervals ranging from weekly to once to three times daily. Alternatively, the peptides disclosed herein may be administered in a cyclical manner (administration of disclosed peptide; followed by no administration; followed by administration of disclosed peptide, and the like). Treatment may continue until the desired outcome is achieved.

The collagen-binding BSP peptide compositions are administered in a therapeutically effective dose in accordance with the invention. A therapeutic concentration will be that concentration which effects the desired level of tissue formation or local tissue repair; or the reduction of a particular condition or the rate of expansion of such condition. A useful therapeutic or prophylactic concentration will vary from condition to condition and in certain instances may vary with the severity of the condition being treated and the patient's susceptibility to treatment. Accordingly, no single concentration may be uniformly useful, but will require modification depending on the particularities of the chronic or acute condition being treated. Such concentrations can be arrived at through routine experimentation as is known to those of skill in the art.

In general, pharmaceutical formulations will include a peptide(s) of the present invention in combination with a pharmaceutically acceptable vehicle, such as saline, buffered saline, 5% dextrose in water, ethanol, borate-buffered saline containing trace metals or the like and mixtures thereof. Formulations may further include one or more excipients, preservatives, solubilizers, buffering agents, albumin to prevent protein loss on vial surfaces, lubricants, fillers, stabilizers, etc. Methods of formulation are well known in the art and are disclosed, for example, in Remington's Pharmaceutical Sciences, Gennaro, ed., Mack Publishing Co., Easton Pa., 1990, which is incorporated herein by reference.

The compositions of the present invention can be used concomitantly with other agents for treating bone diseases. Examples of drugs concomitantly used may include for example, antibiotics, chemotherapeutic agents, enzymes, calcium preparations (e.g. calcium phosphate, calcium sulfate, calcium carbonate), calcitonin preparations, sex hormones (e.g. estrogen, estradiol), prostaglandin A1, bisphosphonic acids, ipriflavones, fluorine compounds (e.g. sodium fluoride), vitamin K, fibrin, proteoglycans, bone morphogenetic proteins (BMPs), fibroblast growth factor (FGF), platelet-derived growth factor (PDGF), transforming growth factor (TGF-β), insulin-like growth factors 1 and 2 (IGF-1, 2), endothelin, parathyroid hormone (PTH), epidermal growth factor (EGF), leukemia inhibitory factor (LIP), osteogenin, and bone resorption repressors such as estrogens, calcitonin and biphosphonates. It is also contemplated that mixtures of such agents may also be used and formulated within the compositions of the present invention or used in conjunction with the compositions of the present invention.

Pharmaceutical compositions for use within the present invention can be in the font of sterile, non-pyrogenic liquid solutions or suspensions, coated capsules, creams, lotions, lyophilized powders or other forms known in the art. The peptides of the invention may be formulated into a hydrogel for local administration or for application to a desired carrier. Local administration may be by injection at the site of injury or defect, or by insertion or attachment of a solid carrier at the site. For local administration, the delivery vehicle may provide a matrix or scaffold for the in-growth of bone or cartilage, and may be a vehicle that can be absorbed by the subject without adverse effects.

A variety of polymers can be used to form an implant for the purposes of delivering the peptide composition of the invention to a desired in vivo site. Suitable polymers include but are not limited to polyesters, polyvinyl acetate, polyacrylates, polyorthoesters, polyhydroxyethylmethacrylate (polyhema), polyanhydrides and chitosan. Certain of the polymers can be selected based on the properties of being both biodegradable and biocompatible. Aliphatic polyesters derived from lactide, glycolide and caprolactone monomers are especially favourable since they possess a fairly broad range of degradation profiles. The peptide compositions of the invention may be used in conjunction with collagen, fibrins, starches, elastin, alginate, and hyaluronic acid.

In addition to the polymers and carriers noted above, the biodegradable films and matrices incorporating the peptide compositions may include other active or inert components and mixtures thereof as discussed supra. Of particular interest are those agents that promote tissue growth or infiltration, such as growth factors. Exemplary growth factors for this purpose include epidermal growth factor (EGF), fibroblast growth factor (FGF), platelet-derived growth factor (PDGF), transforming growth factors (TGFs), parathyroid hormone (PTH), leukemia inhibitory factor (LIF), insulin-like growth factors (IGFs) and the like. Agents that promote bone growth, such as bone morphogenetic proteins (U.S. Pat. No. 4,761,471), osteogenin (Sampath et al. Proc. Natl. Acad Sci USA (1987) 84:7109-13) and NaF (Tencer et al. J. Biomed. Mat. Res. (1989) 23: 571-89) are also preferred. Biodegradable films or matrices include calcium sulfate, tricalcium phosphate, hydroxyapatite, polylactic acid, polyanhydrides, bone or dermal collagen, pure proteins, extracellular matrix components and the like and combinations thereof. Such biodegradable materials may be used in combination with non-biodegradable materials (for example polymer implants, titanium implants), to provide desired biological, mechanical, cosmetic, or matrix interface properties.

In one aspect, the delivery of the peptides described herein to desired sites may be enhanced by the use of controlled-release compositions, such as those described in WIPO publication WO 93/20859 (which is incorporated herein by reference in its entirety). Films of this type are particularly useful as coatings for both resorbable and non-resorbable prosthetic devices and surgical implants. The films may, for example, be wrapped around the outer surfaces of surgical screws, rods, pins, plates and the like. Implantable devices of this type are routinely used in orthopedic surgery. The films can also be used to coat bone-filling materials, such as hydroxyapatite blocks, demineralized bone matrix plugs, collagen matrices and the like. In general, a film or device as described herein is applied to the bone at the fracture site. Application is generally by implantation into the bone or attachment to the surface using standard surgical procedures.

Alternative methods for delivery of compounds of the present invention include use of ALZET osmotic minipumps (Alza Corp., Palo Alto, Calif.); sustained release matrix materials such as those disclosed in Wang et al. (PCT Publication WO 90/11366); electrically charged dextran beads, as disclosed in Bao et al. (PCT Publication WO 92/03125); collagen-based delivery systems, for example, as disclosed in Ksander et al. Ann. Surg. (1990) 211 (3):288-94; methylcellulose gel systems, as disclosed in Beck et al. J. Bone Min. Res. (1991) 6(11): 1257-65; alginate-based systems, as disclosed in Edelman et al. Biomaterials (1991) 12:619-26 and the like. Other methods well known in the art for sustained local delivery in bone include porous coated metal prostheses that can be impregnated and solid plastic rods containing therapeutic compositions of the present invention.

In one embodiment, the peptides may be provided as a solution or emulsion contained within phospholipid vesicles called liposomes. The liposomes may be unilamellar or multilamellar and are formed of constituents selected from phosphatidylcholine, dipalmitoylphosphatidylcholine, cholesterol, phosphatidylethanolamine, phosphatidylserine, demyristoylphosphatidylcholine and combinations thereof. The multilamellar liposomes comprise multilamellar vesicles of similar composition to unilamellar vesicles, but are prepared so as to result in a plurality of compartments in which the selected peptide in solution or emulsion is entrapped. Additionally, other adjuvants and modifiers may be included in the liposomal formulation such as polyethylene glycol, or other materials. It is understood by those skilled in the art that any number of liposome bilayer compositions can be used in the composition of the present invention. Liposomes may be prepared by a variety of known methods such as those disclosed in U.S. Pat. No. 4,235,871 and in RRC, Liposomes: A Practical Approach. IRL Press, Oxford, 1990, pages 33-101. The liposomes containing the peptides of the invention may have modifications such as having non-polymer molecules bound to the exterior of the liposome such as haptens, enzymes, antibodies or antibody fragments, cytokines and hormones and other small proteins, polypeptides or non-protein molecules which confer a desired enzymatic or surface recognition feature to the liposome. Surface molecules which preferentially target the liposome to specific organs or cell types include for example antibodies that target the liposomes to cells bearing specific antigens. Techniques for coupling such molecules are well known to those skilled in the art (see for example U.S. Pat. No. 4,762,915). Alternatively, or in conjunction, one skilled in the art would understand that any number of lipids bearing a positive or negative net charge may be used to alter the surface charge or surface charge density of the liposome membrane. For systemic application by intravenous delivery, it may be beneficial to encapsulate the peptides of the invention within sterically-stabilized liposomes which exhibit prolonged circulation time in blood. The sterically stabilized liposomes are produced containing polyethylene glycol as an essential component of their surface and the method of making such liposomes is known to those skilled in the art.

It is understood by those skilled in the art that other types of encapsulants may also be used to encapsulate collagen-binding BSP peptides of the invention. Microspheres including but not limited to those composed of ion-exchange resins, crystalline ceramics, biocompatible glass, latex and dispersed particles are suitable for use in the present invention. Similarly, nanospheres and other lipid, polymer or protein materials can also be used.

The invention also provides methods for the screening and identifying further functional analogues of the collagen-binding BSP peptides where such identified peptides have essentially the activity of the peptides as described herein. Such screening involves the use of the collagen-binding assay described herein. Screening may also involve contacting a biological sample that is capable of undergoing mineralization with a test peptide or compound and then separately contacting a second biological sample that is also capable of undergoing mineralization with an amount of one or more of the collagen-binding peptides of the invention. The level of mineralization is then assessed by the analysis of one or more criteria selected from the group consisting of bone mineral content, bone nodule mineralization, and collagen assay. The levels of mineralization and bone formation are then compared in each biological sample in order to identify whether the test peptide or compound has essentially the activity of the collagen-binding peptides of the invention. Using a similar approach, modulators of the bone and/or cartilage formation can also be assessed.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

The examples are described for the purposes of illustration and are not intended to limit the scope of the invention.

Methods of synthetic chemistry, protein and peptide biochemistry, molecular biology, histology and immunology referred to but not explicitly described in this disclosure and examples are reported in the scientific literature and are well known to those skilled in the art.

### Example 1

### Materials and Experimental Methods

Rat tail tendon type I collagen was prepared as previously described (Hunter G. K. et al., (2001) Journal of Biomedical Materials Research. 55, 496-502). Rat recombinant BSP (rBSP), rBSP-pE1, 2D and rBSP-pE1,2A (the two contiguous poly[E] sequences of rBSP are mutated to aspartic acid and alanine residues respectively and rBSP(43-101) were expressed in E. coli and purified as previously described (Tye C. E. et al., (2003) J. Biol. Chem. 278).

Construction, Expression and Purification of rBSP Peptides

Partial-length BSP polypeptides incorporating amino acid (1-75), (1-100), (19-100), (99-201) or (200-301) were cloned by the introduction of novel restriction sites by overlap extension PCR (Pogulis R. J. et al., (1996) Methods in Molecular Biology. 57, 167-176), with the incorporation of a 6×His-tag to the carboxyl terminus of the cDNA. Previous studies have shown that the poly-His tag does not interact with collagen (Tye et al; 2005 Identification of the type I collagen-binding domain of bone sialoprotein and the mechanism of interaction. J Biol Chem. February 8; [Epub ahead of print]). The resulting peptides were subcloned into the pET28a expression vector (Novagen). All constructs were confined by DNA sequencing.

The rBSP peptides were expressed in E. coli strain BL21(DE3) cells and were purified by nickel affinity, ion-exchange and size-exclusion chromatography following established protocols (Tye et al., J. Biol. Chem. 278, 7949-7955). Proteins were analyzed for purity and protein content by SDS-PAGE and amino acid analysis.

Alternatively, peptides corresponding to NGVFKYRPRYFLYK (residues 19-32), HAYFYPPLKRFPVQ (residues 33-46) and NGVFKYRPRYFLYKHAYFYPPLKRFPVQ (residues 19-46) of rat BSP were synthesized and purified as described (Fields et al., 1990, Int J Pep Pro Res 35, 161-214; Pampena et al., 2004, Biochem J 378, 1083-1087) and included an N-terminal biotin label. Peptides were purified by analytical HPLC to >95% purity and their identity confirmed by electrospray ionization mass spectrometry (Micromass Quattro II).

### Solid-Phase Collagen-Binding Assay

Binding of proteins to type I collagen was studied by a modification of the method described (Tye et al., (2004) Proc. of the ICCBMT; Calderwood D. A. et al., (1997 Journal of Biological Chemistry. 272, 12311-12317). Type I collagen (1 μg/100 μl) in PBS was plated overnight at 4° C. in 96-well Maxisorp microtitre plates (Nunc). Wells were blocked with 1% myoglobin for 2 hours at 37° C. and incubated with the 6×His-tagged proteins for 3 hours at room temperature. The wells were incubated overnight at 4° C. with 1/2000 Penta-His Antibody (Qiagen) and were subsequently incubated at room temperature for 45 minutes with 1/4000 horseradish peroxidase-conjugated goat anti-mouse IgG (Sigma). The bound proteins were then detected using 0.4 mg/mL ortho-phenylenediamine (Sigma) in phosphate-citrate buffer pH 5 (100 mM citric acid, 200 mM Na₂HPO₄) and 0.012% H₂O₂. The reaction was stopped after 6 minutes by the addition of 2.5 M H₂SO₄ and the absorbance read at 492 nm. Experiments were performed with each concentration in triplicate and data were fitted to a one-site binding model and K_{D} values determined using GraphPad Prism™.

### Characterization of the Interaction Between Type I Collagen and BSP

The solid-phase collagen-binding assay was varied to examine the role of electrostatic interactions in the binding of rBSP and rBSP(1-100) to collagen. The effect of varied pH on binding was examined using 0.1 M phosphate, adjusted to pH 2.5-8.0, as the buffer during the protein incubation and in all washes. Similarly, the effect of ionic strength was investigated by 25 mM Tris, pH 7.0 containing 0-1000 mM NaCl as the buffer. The effect of divalent and trivalent cations on collagen binding was examined using increasing concentrations of CaCl₂, MnCl₂ and LaCl₃ in PBS during protein binding. The A492 of 50 nM rBSP or rBSP(1-100) in the absence of NaCl or cation at pH 7.0 was set to 100% and the percentage of protein bound with increasing ionic strength, decreasing pH or increasing cation concentration was plotted.

Competition of rBSP Binding to Type I Collagen

As a measure of specificity of binding, the ability of unlabelled proteins to compete with biotinylated protein was assessed. RBSP and rBSP(1-100) were biotinylated with EZ-Link™ Sulfo-NHS-Biotin (Pierce) following the manufacturer's protocol. Labeled rBSP or rBSP(1-100) plus various concentrations of unlabeled protein were incubated with collagen for 3 hours as above. Following the collagen incubation, the wells were incubated with 10 μg/mL Extravidin Peroxidase (Sigma) in TBS for 15 minutes at room temperature. The bound protein was then detected as above with ortho-phenylenediamine in phosphate-citrate buffer. The A492 of biotinylated rBSP in the absence of competitor was set to 100% and the percentage of biotinylated rBSP bound with increasing competitor was plotted. The competition assays included 10 nM biotinylated rBSP incubated with 0-1000 nM of unlabeled rBSP-pE1,2D, rBsp-pE1,2A; 25 nM rBSP incubated with 0-1000 nM of unlabeled rBSP(1-100), rBSP(99-201) or rBSP(200-301); and 50 nM rBSP(1-100) incubated with 0-1000 nM of unlabeled rBSP(1-100), rBSP(1-75), rBSP(19-100) or rBSP(43-101).

### Circular Dichroism Spectroscopy

The effect of pH and ionic strength on the conformation of rBSP(1-75) was studied by circular dichroism spectroscopy (CD). The far-UV spectra of rBSP(1-75) was recorded in quartz cells of 1 mm optical path length using a Jasco-J810 spectropolarimeter between 190 and 260 nm, in 0.5 nm steps. The protein was studied at 0.2 mg.ml in buffers containing 0.1 M phosphate with pH 2.5-8.0 or 25 mM Tris-HCl, pH 7.0 with 0-1000 mM NaCl, as used above. A baseline with buffer only was recorded separately and subtracted from each spectrum. All spectra were recorded at room temperature. The molar ellipticity (θ) expressed in degrees cm² dmol⁻¹ was calculated on the basis of mean residue molecular mass.

Estimates of protein secondary structure from the CD data were made using the Circular Dichroism Deconvolution by Backpropagation Neural Networks (CDNN) program (25) as well as a calculation from [φ]220 ₙₘ.

### Example 2

### Electrostatic Interactions in the Binding of rBSP to Type I Collagen

No difference in rBSP binding was observed when using the Tris or phosphate buffers at physiological pH (data not shown). The binding of rBSP to collagen was reduced, however, by increasing the ionic strength of the buffer (FIG. 3A). Similarly, binding to collagen decreased at lower pH values (FIG. 3B). Increasing concentrations of CaCl₂, MnCl_{2 or} LaCl₃ caused concentration-dependent decreases in binding (FIG. 3C). In all of the above cases, binding was reduced indicating an electrostatic component to the binding of BSP with collagen. However, 25-45% of the protein still remained bound at high salt or low pH, implying that binding was not entirely electrostatic.

### Example 3

### Role of the Poly Glutamic Acid in Collagen Binding

rBSp, rBSP-pE1,2D and rBSP-pE1,2A were tested for collagen-binding activity to examine the contributions of the contiguous glutamic acid residues to collagen binding. rBSP, rBSP-pE1,2A and rBSP-pE1,2D demonstrate saturable binding to type I collagen (FIG. 4A). Based on a one-site binding model, rBSP has a K_{D}=22.85±3.9 nM. Confirmation of the specificity of these mutated proteins for collagen is evident by the ability of these mutant proteins to compete with labelled rBSP for binding to collagen (FIG. 4B).

### Example 4

### Localization of the Collagen-Binding Domain on rBSP

To locate the collagen-binding domain of rBSP, several peptides were expressed and tested for collagen-binding activity. Both rBSP(99-201) and rBSP(200-301) show negligible binding to collagen (FIG. 5A), which is confirmed by the inability to either peptide to compete with the binding of rBSP to collagen (FIG. 5B). rBSP(1-100), however, bound to collagen with a K_{D}=5.64+0.59 nM, which is an increased affinity compared to the full-length protein (K_{D}=22.85+3.9 nM). This strong affinity for collagen is evident by the ability of rBSP(1-100) to compete with rBSP for binding (FIG. 5B). rBSP(1-75) shows binding comparable to rBSP(1-100) with a K_{D}=4.04+0.59 nM; however, the binding of rBSP(19-100) was somewhat_lower, with K_{D}=50.26+9.46 nM (FIG. 5C). The specificity of these two peptides was demonstrated by their ability to compete for binding with rBSP(1-100) as seen in FIG. 5D. rBSP(43-101) was not tested for binding activity, however, it was not able to compete for binding with rBSP(1-100) which indicates that the binding-domain is not within residues 43-101 (FIG. 5D), but may still nevertheless involve the first 3 to 4 amino acids (43-46).

### Example 5

### Effect of Varying pH and Ionic Strength on Conformation of rBSP(1-75)

In either 0.1 M phosphate or 25 mM Tris buffer, the conformations as determined by CD analysis of rBSP(1-75) at pH 7.0 are equivalent (data not shown). Increasing concentrations of NaCl do not alter these conformations as the spectra are identical (data not shown). Decreasing the pH of the buffer, however, does alter the conformation of the protein slightly and a shift of the minima to the right is seen (FIG. 6). This right shift is indicative of an increase in α-helical content. Secondary structure estimates by the CDNN program estimates that rBSP(1-75) at pH_{—}7.0 exhibits 5.9% α-helix, 36.4% anti-parallel β-sheet, 3.2% parallel β-sheet, 20.2% β-turn and 34.1% unordered structure. This conformation is stable to about pH 4 when the α-helical content increases to 6.2%, 6.9% at pH 3.5, and 7.5% at pH 2.5. The other secondary-structure elements are unchanged. Calculation of secondary structure from [Φ]₂₂₀ nm gives different percentages; however, it shows the same trend of increasing α-helical content. By this method, α-helical content is 1.9% at pH 7.0 and begins to increase at pH 4.5 to 2.3%, 3.0% at pH 4.0, 5.6% at pH 3.5 and 6.2% at pH 2.5.

### Example 6

### Animal Studies

To test the potential and applicability of the novel collagen binding sequences of the invention, peptides containing such a sequence were administered to repair defects in rat calvariae. The protocol that was utilized is based on accepted bone-repair models, which involves analysis of repair in critical-sized defects generated in rat calvariae. Holes of sufficient size are considered to be critical sized as they will normally not be repaired without some type of intervention. The surgical protocol was approved by the University of Western Ontario Council on Animal Care. Male Wistar rats (about 80-days old and approximately 280 g) were used. A 3-cm incision was made along the sagittal suture to reflect the skin. One full-thickness defect of 6 mm diameter was made in the centre of the parietal bone with a trephine drill under constant irrigation (Ringer's salt solution). An absorbable collagen sponge (6 mm diameter) was immersed in test protein in sterile saline solution for at least 30 minutes (controls are vehicle alone), then inserted into the hole and the wound sutured. To determine if mineralization of the defect was occurring, the animals were anesthetized and subjected to micro-computerized tomography (μ-CT) imaging using a GE RS-80 In Vivo Scanner at day 11 and day 33 post-surgery. The scanner has an approximately 50 μm resolution and is routinely used to measure bone mineral density. An internal control of hydroxyapatite placed on each animal was used to calibrate each scan. Bone mineral content was determined and shown in Table 1, and scanned images of these rat calvariae at day 33 shown in FIG. 7. These results indicate that the peptides of the invention as administered are effective in repair of bone defects.

Table 1. Bone mineral content (total mg) of critical sized defects that have implanted resorbable collagen sponges impregnated with buffer alone or with test BSP peptide reagents. The entire rat calvaria was scanned, bone mineral density and thus bone mineral content (BMC) was determined for both the entire 6-mm diameter defect as well as a 5.5-mm diameter to exclude the perimeter of the defect.

| | BMC for 6 mm area | | BMC for 5.5 mm area | |
|---|---|---|---|---|
| | 11 days | 33 days | 11 days | 33 days |
| Control (vehicle alone) | 0.744 | 2.029 | 0.382 | 1.491 |
| Control (vehicle alone) | 0.713 | 1.887 | 0.382 | 1.454 |
| Native rat BSP | 1.024 | 5.010 | 0.692 | 3.857 |
| Native rat BSP | 0.646 | 4.066 | 0.284 | 3.233 |
| rBSP1-100 | 0.675 | 6.239 | 0.453 | 5.448 |
| rBSP1-100 | 1.201 | 4.492 | 0.815 | 3.859 |

Although preferred embodiments of the invention have been described herein in detail, it will be understood by those skilled in the art that variations may be made thereto without departing from the spirit of the invention.

1. A collagen-binding BSP peptide comprising a portion of the N-terminal domain of the BSP protein sequence.

2. The peptide of claim 1, wherein said peptide is essentially hydrophobic and wherein said peptide binds to type I collagen.

3. The peptide of claim 1, wherein said BSP protein sequence is selected from the group consisting of a rat BSP sequence, a mouse BSP sequence, a human BSP sequence, a bovine BSP sequence, a porcine BSP sequence and a hamster BSP sequence.

4. The peptide of claim 1, wherein said peptide is up to about 28 amino acids in length.

5. The peptide of claim 4, wherein said peptide has the amino acid sequence NGVFKYRPRYFLYKHAYFYPPLKRFPVQ.

6. The peptide of claim 4, wherein said peptide has an amino acid sequence selected from the group consisting of NGVFKYRPRYFLYK-Z, Z-HAYFYPPLKRFPVQ and NGVFKYRPRYFLYKHAYFYPPLKRFPVQ and functional analogues thereof.

7. A fragment of the peptide of claim 1.

8. A functional analogue of the peptide of claim 1.

9. The functional analogue of claim 8, wherein said functional analogue comprises a peptide conjugate, a chimeric protein or a peptide mimetic.

10. The fragment of claim 7, wherein said fragment is four or more amino acids in length.

11. The peptide of claim 1, wherein said peptide further comprises one or more linker groups.

12. The peptide of claim 11, wherein said linker group is selected from the group consisting of carbodiimide, aldehyde, maleimide, sulfhydryl, amino, carboxy, hydroxy and NHS esters, a modified cysteine, a phosphorylated amino acid, an amino acid, diacetic acid, sulfonyl chloride, isocyanate, isothiocyanate, epoxy, bisphosphonate, pyrophosphate, phosphate, disulfide, phenyl azide, alkyl halide and hydrazide acyl chloride.

13. The peptide of claim 12, wherein said linker group is proteolytically cleavable.

14. The peptide of claim 1, wherein said peptide is provided as a composition with a pharmaceutically acceptable carrier.

15. The peptide of claim 14, wherein said composition further comprises an agent selected from the group consisting of calcium phosphate, calcium sulfate, calcium carbonate, fibrin, hyaluronic acid, proteoglycans, calcitonin, estrogen, estradiol, prostaglandin A1, bisphosphonic acids, ipriflavones, sodium fluoride, vitamin K, bone morphogenetic proteins, fibroblast growth factor, platelet-derived growth factor, transforming growth factor, insulin-like growth factors 1 and 2, endothelin, parathyroid hormone, epidermal growth factor, leukemia inhibitory factor, osteogenin and mixtures thereof.

16. The peptide of claim 14, wherein said composition may be administered locally and/or systemically.

17. The peptide of claim 16, wherein local administration is provided by injection or use with an implant.

18. The peptide of claim 1, wherein said peptide is provided as a coating, filling or dispersed within a matrix of an implant.

19. The peptide of claims 1, wherein said peptide is provided with a biocompatible and/or biodegradable polymer.

20. A chimeric protein that stimulates mineralization of tissues, the chimeric protein comprising:
(a) a collagen-binding BSP peptide; and
(b) a HA-nucleating moiety.

21. The chimeric protein of claim 18, wherein (a) is selected from the group consisting of NGVFKYRPRYFLYK-Z, Z-HAYFYPPLKRFPVQ, NGVFKYRPRYFLYKHAYFYPPLKRFPVQ, mixtures thereof and fragments thereof.

22. A composition for stimulating mineralization of a tissue in a mammal, said composition comprising
an effective amount of
(a) a peptide selected from the group consisting of NGVFKYRPRYFLYK-Z, Z-HAYFYPPLKRFPVQ and NGVFKYRPRYFLYKHAYFYPPLKRFPVQ;
(b) a fragment or analogue of any peptide of (a); and
(c) a mixture of any one of (a) and (b), wherein said peptide is conjugated to a bioactive molecule that stimulates mineralization of said tissue.

23. The composition of claim 22, wherein said peptide is a chimeric peptide.

24. A method for stimulating mineralization of a desired tissue expressing type I collagen, the method comprising administering to said tissue an effective amount of a collagen-binding BSP peptide conjugated to a mineralization promoting bioactive molecule.

25. A method for stimulating mineralization of a desired tissue expressing type I collagen, the method comprising administering to said tissue an effective amount of a chimeric collagen-binding BSP peptide, said chimeric peptide further comprising a protein/peptide that stimulates mineralization in said tissue.

26. A method for stimulating bone formation, said method comprising administering an effective amount of:
(a) a collagen-binding BSP peptide conjugated to a mineralization promoting bioactive molecule;
(b) a chimeric collagen-binding BSP peptide, said chimeric peptide further comprising a protein/peptide that stimulates mineralization; and
(c) a combination of (a) and (b).

27. A method for inhibiting mineralization in a tissue, said method comprising administering an effective amount of,
(a) a collagen-binding BSP peptide;
(b) a collagen-binding BSP peptide conjugated with a bioactive molecule that is an inhibitor of mineralization;
(c) a chimeric protein comprising a collagen-binding BSP peptide and an inhibitory HA-nucleating moiety; and
(d) any combination of (a) to (c).

28. The method of claim 24, wherein said peptide is selected from the group consisting of NGVFKYRPRYFLYK-Z, Z-HAYFYPPLKRFPVQ and NGVFKYRPRYFLYKHAYFYPPLKRFPVQ.

29. The use of a collagen-binding BSP peptide comprising a portion of the N-terminal domain of the BSP protein sequence in a medicament for the treatment of a desired tissue expressing Type I collagen.

30. The use of claim 29, wherein said composition is for the stimulation of mineralization of said tissue.

31. The use of claim 29, wherein said composition is for the inhibition of mineralization in a tissue.

32. The peptide of claim 6, wherein Z is selected from one or more amino acids such as but not limited to alanine, arginine, asparagines, aspartate, cysteine, glutamate, glutamine, glycine, histidine, isoleucine, leucine, lysine, methionine, phenylalanine, praline, serine, threonine, tryptophan, tyrosine, valine, hydroxyproline and any other modified amino acid.

33. The composition of claim 22, wherein Z is selected from one or more amino acids such as but not limited to alanine, arginine, asparagines, aspartate, cysteine, glutamate, glutamine, glycine, histidine, isoleucine, leucine, lysine, methionine, phenylalanine, praline, serine, threonine, tryptophan, tyrosine, valine, hydroxyproline and any other modified amino acid.

34. The method of claim 28, wherein Z is selected from one or more amino acids such as but not limited to alanine, arginine, asparagines, aspartate, cysteine, glutamate, glutamine, glycine, histidine, isoleucine, leucine, lysine, methionine, phenylalanine, praline, serine, threonine, tryptophan, tyrosine, valine, hydroxyproline and any other modified amino acid.

35. The method of claim 25, wherein said peptide is selected from the group consisting of NGVFKYRPRYFLYK-Z, Z-HAYFYPPLKRFPVQ and NGVFKYRPRYFLYKHAYFYPPLKRFPVQ.

36. The method of claim 26, wherein said peptide is selected from the group consisting of NGVFKYRPRYFLYK-Z, Z-HAYFYPPLKRFPVQ and NGVFKYRPRYFLYKHAYFYPPLKRFPVQ.

37. The method of claim 27, wherein said peptide is selected from the group consisting of NGVFKYRPRYFLYK-Z, Z-HAYFYPPLKRFPVQ and NGVFKYRPRYFLYKHAYFYPPLKRFPVQ.
